(19) United States
(12) Patent Application Publication
(10) Pub. No.: US 2019/0314269 A1
(43) Pub. Date: Oct. 17, 2019

(54) NOVEL METHOD

(71) Applicant: DSM IP ASSETS B.V., Heerlen (NL)

(72) Inventors: Dominik IMFELD, Kaiseraugst (CH); Rolf SCHUETZ, Kaiseraugst (CH); Eliane Ursula WANDELER, Kaiseraugst (CH)

(21) Appl. No.: 16/343,126

(22) PCT Filed: Oct. 26, 2017
(86) PCT No.: PCT/EP2017/077378

§ 371 Date: Apr. 18, 2019

(30) Foreign Application Priority Data
Oct. 27, 2016 (EP) 16196022.4

(51) Int. Cl. A61K 8/9706; A61K 8/73; A61K 8/55; A61K 8/06; A61Q 17/00

(52) U.S. Cl. CPC A61K 8/9706; A61K 8/73; A61Q 17/00; A61K 8/062; A61K 8/55

(57) ABSTRACT

The present application relates to a method of protecting keratinocytes against the effects of air pollution caused by introduction of dust particles, fumes or smoke into the atmosphere such as in particular against the harmful effects of diesel and urban dust particular matter, said method including applying to the skin a cosmetic composition containing an effective amount of an active selected from the group consisting of pullulan and a nannochloropsis occulata extract as well as mixtures thereof.

The present application relates to a method of protecting keratinocytes against the effects of air pollution caused by introduction of dust particles, fumes or smoke into the atmosphere such as in particular against the harmful effects of diesel and urban dust particular matter, said method including applying to the skin a cosmetic composition containing an effective amount of an active selected from the group consisting of pullulan and a nannochloropsis occulata extract as well as mixtures thereof.

Certain urban environments are regularly subjected to peaks of air pollution, which occurs when gases, dust particles, fumes (or smoke) or odor are introduced into the atmosphere in a way that makes it harmful to humans, animals and plants. Air pollution can result from both human and natural actions. Natural events that pollute the air include forest fires, volcanic eruptions, wind erosion, pollen dispersal, evaporation of organic compounds and natural radioactivity. Pollution from natural occurrences is not very often. The majority of air pollution results from human activities, including: Emissions from industries and manufacturing activities and burning fossil fuels (such as e.g. emissions from automobile engines).

Particulate Matter (PM) is formed through chemical reactions, fuel combustion (e.g., burning coal, wood, diesel), industrial processes, farming (plowing, field burning), and unpaved roads or during road constructions.

An individual in his daily environment in an urban area may thus be subjected to a whole range of factors attacking the skin and thus also the skin cells such as in particular the keratinocytes, by various airborne pollutants such as in particular by particulate material, e.g. diesel and urban dust particles which negatively affect cell survival of keratinocytes.

Thus, there is an ongoing need for cosmetic compositions which protect the keratinocytes from the harmful effect of air pollution such as in particular diesel and urban particular matter.

Surprisingly, it has been found that pullulan and a nannochloropsis occulata extract as well as mixtures thereof effectively protect keratinocytes against the adverse effects of pollutants found in the atmosphere such as in particular against diesel and urban dust particular matter.

Thus, one object of the present invention relates to a method of protecting keratinocytes against the adverse effects of air pollution caused by introduction of dust particles, fumes or smoke into the atmosphere, in particular against the harmful effect of diesel and urban dust particular matter, said method including applying to the skin a cosmetic composition containing an effective amount of an active selected from the group consisting of pullulan and a nannochloropsis occulata extract as well as mixtures thereof, and optionally appreciating the protective effect.

Another object of the invention is the use of an active selected from the group consisting of pullulan and a nannochloropsis occulata extract as well as mixtures thereof to prepare a cosmetic composition for the protection of keratinocytes against the harmful effects of air pollution caused by introduction of dust particles, fumes or smoke into the atmosphere, in particular against the harmful effect of diesel and urban dust particular matter.

A further object of the present invention relates to the cosmetic use of an active selected from the group consisting of pullulan and a nannochloropsis occulata extract as well as mixtures thereof for the protection of keratinocytes against the harmful effects of diesel and urban dust particular matter.

Pullulan is a polysaccharide produced by the yeast like fungus Aureobasidium pullulans. Pullulan is an essentially linear glucan consisting mainly of 1,6-linked maltotriose and some interspersed maltotetraose units. Pullulan is e.g. commercially available as Aqua beta (from Daiso, Co., Ltd.) or Pullulan (from Hayashibara Co., Ltd.).

The nannochloropsis oculata extract [CAS 92128-82-0] is preferably an extract of the biotechnologically produced microalgae nannochloropsis oculata. Such extracts are obtainable by cultivation of the respective algae, followed by harvesting the cells. Afterwards the harvested cells are rehydrated and extracted with hot water. The crude extract is subsequently centrifuged and ultrafiltrated. The such obtained nannochloropsis oculata extract can be used in dried form or in the form of a concentrated aqueous extract with a known solid content, preferably with a solid content selected in the range of 0.5 to 10 wt.-%, preferably in the range of 1 to 5 wt.-%, based on the total aqueous nannochloropsis extract.

In all embodiments of the present invention, the actives are preferably used in the form of an active blend comprising a nannochloropsis occulata extract and pullulan. Particularly suitable active blends according to the present invention are water based blends of a nannochloropsis occulata extract and pullulan. Such active blends preferably consist essentially of >50% of water, 1-5% of a nannochloropsis occulata extract, 10-25% of pullulan and less than 3% of a preservative such as preferably and 0.1-1% of each of phenoxyethanol, sodium benzoate and potassium sorbate. A particularly preferred blend according to the present invention is PEPHA®-TIGHT available form DSM Nutritional Products.

The term ‘an effective amount’ refers to an amount necessary to obtain the physiological effect. The physiological effect may be achieved by one application dose or by repeated applications. The dosage administered may, of course, vary depending upon known factors, such as the physiological characteristics of the particular cosmetic composition comprising the respective active and its mode and route of administration; the age, the nature and extent of the symptoms; the kind of concurrent treatment; the frequency of treatment; and the effect desired and can be adjusted by a person skilled in the art.

The term ‘protecting against the harmful effects’ refers to an increased viability of the keratinocytes compared to keratinocytes which are not treated with the respective active/mixture of actives. In a particular preferred embodiment, the viability of the keratinocytes is increased by at least 3%, more preferably by at least 5%, when compared to non-treated keratinocytes.

The term ‘consist/consisting essentially of’ as used according to the present invention means that the total amount of the listed ingredients ideally sum up to 100 wt.-%. It is however not excluded that small amounts of impurities or additives may be present, with the proviso that the total amount of such impurities or additives is preferably less than 3 wt. %, more preferably less than 2 wt.-%, most preferably less than 1 wt.-% and which are introduced via the respective raw materials.

Thus, in another embodiment, the present invention refers to a method according to the present invention wherein the viability of the keratinocytes is increased by at least 3%, preferably by at least 5%, when compared to non-treated keratinocytes.

The total amount of the active(s) used in the cosmetic composition according to the present invention is generally selected in the range of 0.00001 to 1 wt. %, preferably in the range of 0.00003 to 0.5 wt.-% based on the total weight of the cosmetic composition.

Advantageously, the amount of the active blends according to the present invention with all the definitions and preferences given above, such as in particular Pepha®-Tight, in the cosmetic compositions is selected in the range of 0.01 to 10 wt. %, more preferably in the range of 0.1 to 5 wt. %, most preferably in the range of 0.1 to 3 wt. %, based on the total weight of the cosmetic composition.

Preferably, the amount of the cosmetic composition to be applied to the skin is selected in the range of 0.1 to 3 mg/cm² skin, such as preferably in the range of 0.1 to 2 mg/cm² skin and most preferably in the range of 0.5 to 2 mg/cm² skin.

The term ‘cosmetic composition’ as used herein refers to compositions, which are used to treat, care for or improve the appearance of the skin and/or the scalp. Particular advantageous cosmetic compositions are skin care preparations.

The cosmetic compositions according to the invention are intended for topical application, which is to be understood as the external application to keratinous substances, such as in particular the skin.

As the cosmetic compositions according to the invention are intended for topical application, they comprise a physiologically acceptable medium, that is to say a medium compatible with keratinous substances, such as in particular the skin. In particular the physiologically acceptable medium is a cosmetically, respectably pharmaceutically acceptable carrier.

The term ‘cosmetically acceptable carrier’ respectively ‘pharmaceutically acceptable carrier’ as used herein refers to a physiologically acceptable medium which is compatible with keratinous substances. Suitable carriers are well known in the art and are selected based on the end-use application. Preferably, the carriers of the present invention are suitable for application to skin (e.g., sunscreens, creams, milks, lotions, masks, serums, hydrodispersions, foundations, creams, creamgels, or gels etc.). Such carriers are well-known to one of ordinary skill in the art, and can include one or more compatible liquid or solid filler diluent, excipient, additive or vehicle which are suitable for application to skin. The exact amount of carrier will depend upon the level of the active(s), respectively active blends and any other optional ingredients that one of ordinary skill in the art would classify as distinct from the carrier (e.g., other active components). The cosmetic compositions of the present invention preferably comprise from about 75% to about 99.999%, more preferably from about 85% to about 99.99%, still more preferably from 90% to about 99%, and most preferably, from about 93% to about 98%, by weight of the cosmetic composition, of a carrier.

The cosmetic compositions of the present invention can be formulated into a wide variety of product types, including creams, waxes, pastes, lotions, milks, mousses, gels, oils, tonics, and sprays. Preferably the active(s), respectively the active blends are formulated into lotions, creams, gels, and tonics. These product forms may be used for a number of applications, including, but not limited to, hand and body lotions, facial moisturizers, anti-ageing preparations, make-ups including foundations, and the like. Any additional components required to formulate such products vary with product type and can be routinely chosen by one skilled in the art.

If cosmetic compositions of the present invention are formulated as an aerosol and applied to the skin as a spray-on product, a propellant is added to the cosmetic composition.

The cosmetic compositions according to the present invention can be prepared by conventional methods in the art such as e.g. by admixing an active or an active blend according to the present with the cosmetically acceptable carrier.

The cosmetic compositions of the invention (including the carrier) may comprise further conventional adjuvants and additives, such as preservatives/antioxidants, fatty substances/oils, water, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such cosmetic compositions.

In accordance with the present invention, the cosmetic compositions according to the invention may also comprise further cosmetically active ingredients conventionally used in cosmetic and/or pharmaceutical compositions. Exemplary active ingredients encompass skin lightening agents; UV-filters, agents for the treatment of hyperpigmentation; agents for the prevention or reduction of inflammation; firming, moisturizing, soothing, and/or energizing agents as well as agents to improve elasticity and skin barrier.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

The necessary amounts of further active ingredients as well as the excipients, diluents, adjuvants, additives etc. can, based on the desired product form and application, easily be determined by the skilled person. The additional ingredients can either be added to the oily phase, the aqueous phase or separately as deemed appropriate.

The further cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The cosmetic compositions according to the present invention may be in the form of a suspension or dispersion in solvents or fatty substances, or alternatively in the form of an emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. oil-in-water-in oil (O/W/O) or water-in-oil-in-water (W/O/W) type), pickering emulsion, hydrogel, alcoholic gel, lipogel, one- or multiphase solution or vesicular dispersion or other usual forms, which can also be applied by pens, as masks or as sprays.

If the cosmetic composition is an emulsion, such as in particular an O/W, W/O, Si/W, W/Si, O/W/O, W/O/W multiple or a pickering emulsion, then the amount of the oily phase present in such cosmetic emulsions is preferably at least 10 wt.-%, such as in the range of 10 to 60 wt.-%, preferably in the range of 15 to 50 wt.-%, most preferably in the range of 15 to 40 wt.-%, based on the total weight of the cosmetic composition.

In one embodiment, the cosmetic compositions according to the present invention are advantageously in the form of an oil-in-water (O/W) emulsion comprising an oily phase dispersed in an aqueous phase in the presence of an O/W emulsifier. The preparation of such O/W emulsions is well known to a person skilled in the art.

If the cosmetic composition according to the invention is an O/W emulsion, then it contains advantageously at least one O/W- or Si/W-emulsifier selected from the list of, glyceryl stearate citrate, glyceryl stearate SE (self-emulsifying), stearic acid, salts of stearic acid, polyglyceryl-3-methylglycosedistearate. Further suitable emulsifiers are phosphate esters and the salts thereof such as cetyl phosphate (e.g. as Amphisol® A from DSM Nutritional Products Ltd.), diethanolamine cetyl phosphate (e.g. as Amphisol® DEA from DSM Nutritional Products Ltd.), potassium cetyl phosphate (e.g. as Amphisol® K from DSM Nutritional Products Ltd.), sodium cetearylsulfate, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, cetearyl glucoside, lauryl glucoside, decyl glucoside, sodium stearoyl glutamate, sucrose polystearate and hydrated polyisobutene. Furthermore, one or more synthetic polymers may be used as an emulsifier. For example, PVP eicosene copolymer, acrylates/C10-30 alkyl acrylate crosspolymer, and mixtures thereof.

The at least one O/W, respectively Si/W emulsifier is preferably used in an amount of 0.5 to 10 wt. %, in particular in the range of 0.5 to 6 wt.-%, such as more in particular in the range of 0.5 to 5 wt.-%, such as most in particular in the range of 1 to 4 wt.-%, based on the total weight of the cosmetic composition.

Particular suitable O/W emulsifiers to be used in the cosmetic compositions according to the invention encompass phosphate ester emulsifiers such as advantageously 8-10 alkyl ethyl phosphate, C9-15 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate.

A particular suitable O/W emulsifier to be used in the cosmetic compositions according to the invention is potassium cetyl phosphate e.g. commercially available as Amphisol® K at DSM Nutritional Products Ltd Kaiseraugst.

Another particular suitable class of O/W emulsifiers are non-ionic self-emulsifying systems derived from olive oil e.g. known as (INCI Name) cetearyl olivate and sorbitan olivate (chemical composition: sorbitan ester and cetearyl ester of olive oil fatty acids) sold under the tradename OLIVEM 1000.

In one particular embodiment, the invention relates to cosmetic compositions with all the definitions and preferences given herein in the form of O/W emulsions comprising an oily phase dispersed in an aqueous phase in the presence of an O/W emulsifier wherein the O/W emulsifier is potassium cetyl phosphate. The amount of oily phase in such O/W emulsions is preferably at least 10 wt.-%, more preferably in the range of 10 to 60 wt.-%, most preferably in the range of 15 to 50 wt.-%, such as in the range of 15 to 40 wt.-%.

The cosmetic compositions according to the invention in general have a pH in the range of 3 to 10, preferably a pH in the range of 4 to 8 and most preferably a pH in the range of 4 to 7.5. The pH can easily be adjusted as desired with suitable acids, such as e.g. citric acid, or bases, such as sodium hydroxide (e.g. as aqueous solution), triethanolamine (TEA Care), Tromethamine (Trizma Base) and Aminomethyl Propanol (AMP-Ultra PC 2000), according to standard methods in the art.

The following examples are provided to further illustrate the effects of the present invention. These examples are illustrative only and are not intended to limit the scope of the invention in any way.

### 1. Description of Particulate Matter Assay

As authentic surrogates for urban air pollution we used Standard Reference Material® (SRM) 1649b (“Urban Particulate Matter) of the National Institute of Standards and Technology (Gaithersburg, Md., USA). The particulate matter SRM1649b was resuspended in Dimethylsulfoxid (DMSO), vigorously shaken by vortex and then stirred for 1 hour. After centrifugation, the supernatant was added to cell culture (0.3 mg/ml final), which contained PEPHA® Tight (DSM Nutritional Products Ltd) in the concentration as indicated in the table below. We used primary human epidermal keratinocytes (CellNTec Ltd., Bern, Switzerland) that were cultured in serum-free keratinocyte growth medium CnT-PR at 37° C. in a humidified 5% CO₂-air atmosphere. Cells were subcultured before reaching confluence. For the evaluation of viable cells in culture we used the PrestoBlue Cell Viability Reagent from Thermo Fisher Scientific. PrestoBlue is a resazurin-based solution that turns red and highly fluorescent by the reducing power of living cells. All assays were done in triplicate. The results are presented in the table below as cellular viability in percentage (%) versus control, i.e. medium only, which value was set to 100%. The results illustrate that respective actives effectively counteract the negative effect of the urban particulate matter.

### Results of Particulate Matter Assay

TABLE 1
Cell viability after Urban Particulate Matter
(SRM1649b) exposure: PEPHA-THIGHT

| | | 2 | 3 |
|---|---|---|---|
| Medium | 1 (Reference) | 0.01% PEPHA ® | 0.10% PEPHA ® |
| only | — | Tight | Tight |
| 100% | 53.4% | 67.7% | 68.2% |

### 2. Exemplary Cosmetic Composition to Protect Keratinocytes Against the Harmful Effects of Air Pollution Caused by Introduction of Dust Particles, Fumes or Smoke into the Atmosphere

| O/W Emulsions | Wt-% | Wt.-% | Wt.-% |
|---|---|---|---|
| Glyceryl Stearate | 1 | 1 | 1 |
| PEG-100 Stearate | | 1 | 1 |
| Potassium Cetyl Phosphate | 3 | 1.5 | 1.5 |
| Cetearyl Alcohol | 2 | | |
| Cetyl Alcohol | | 0.5 | 0.5 |
| Acrylates/C₁₀₋₃₀ Alkyl Acrylate | 0.2 | | |
| Crosspolymer | | | |
| Xanthan Gum | | 0.3 | 0.3 |
| C₁₂₋₁₅ Alkyl Benzoate | 10 | 5 | 5 |
| Butylene Glycol Dicaprylate/Dicaprate | | 9 | 9 |
| Hydrogenated Polydecene | | 2 | 2 |
| Caprylic/Capric Triglyceride | 5 | | |
| Methylpropanediol | | 3 | 3 |
| Glycerine | 5 | 3 | 3 |
| Glyceryl Glucoside | | 2 | 2 |
| Alcohol denat. | | 4 | 4 |
| Isotridecylsalicylate | 3 | 5 | 5 |
| PEPHA ® Tight | 1 | 3 | 0.1 |
| Ethylhexyl Benzoate | 1.5 | 1 | 1 |
| Butyl Methoxydibenzoylmethane | 3 | 3 | 3 |
| Octocrylene | 10 | | |
| Bis-Ethylhexyloxyphenol | | 1 | 1 |
| Methoxyphenyltriazine | | | |
| Methyl Methacrylate Crosspolymer | 2 | | |
| Fragrance, Preservatives | q.s. | q.s. | q.s. |
| Sodium Hydroxide | q.s. | q.s. | q.s. |
| Water | Ad 100 | Ad 100 | Ad 100 |

1. A non-therapeutic cosmetic method of protecting keratinocytes against the harmful effects of air pollution caused by introduction of dust particles, fumes or smoke into the atmosphere, said method including applying to the skin a cosmetic composition containing an effective amount of an active selected from the group consisting of pullulan and a nannochloropsis occulata extract as well as mixtures thereof.

2. The method according to claim 1 wherein the harmful effect is caused by diesel and urban dust particulate matter.

3. The method according to claim 1, wherein the effective amount of the active(s) (total) is selected in the range of 0.00001 to 1 wt.-%, preferably in the range of 0.00003 to 0.5 wt.-% based on the total weight of the composition.

4. The method according to claim 1, wherein the amount of the cosmetic composition to be applied to the skin is selected in the range of 0.1 to 3 mg/cm² skin,

5. The method according to claim 1, wherein the amount of the cosmetic composition to be applied to the skin is selected in the range of 0.1 to 2 mg/cm² skin.

6. The method according to claim 1, wherein the amount of the cosmetic composition to be applied to the skin is selected in the range of 0.5 to 2 mg/cm² skin.

7. The method according to claim 1, wherein the actives are used in the form of an active blend consisting essentially of >50% of water, 1-5% of a nannochloropsis occulata extract, 10-25% of pullulan and less than 3% of a preservative.

8. The method according to claim 1, wherein the cosmetic composition is a skin care preparation.

9. The method according to claim 1, wherein the cosmetic composition is an O/W emulsion comprising an oily phase dispersed in an aqueous phase in the presence of an O/W emulsifier.

10. The method according to claim 9, wherein the O/W emulsifier is potassium cetyl phosphate.

11. Use of an active selected from the groups consisting of pullulan and a nannochloropsis occulata extract as well as mixtures thereof to prepare a cosmetic composition for the protection of keratinocytes against the harmful effects of air pollution caused by introduction of dust particles, fumes or smoke into the atmosphere.

12. The use according to claim 11, for the protection against the harmful effects of diesel and urban dust particulate matter.

13. Pullulan or a nannochloropsis occulata extract as well as mixtures thereof for use in the protection of keratinocytes against the harmful effect of diesel and urban dust particulate matter.

14. Pullulan or a nannochloropsis occulata extract as well as mixtures thereof for the use according to claim 13, wherein the actives are used in the form of an active blend consisting essentially of >50% of water, 1-5% of a nannochloropsis occulata extract, 10-25% of pullulan and less than 3% of a preservative is used.

15. Pullulan or a nannochloropsis occulata extract as well as mixtures thereof for the use according to claim 13, wherein the viability of the keratinocytes is increased by at least 3%.
